(19) United States
(12) Patent Application Publication
(10) Pub. No.: US 2008/0138469 A1
(43) Pub. Date: Jun. 12, 2008

(54) Novel vitamin D2 yeast preparation, a method for producing the same, and the use thereof

(75) Inventors: Richard Degre, St-Bruno (CA); Zhigen Zhang, Notre-Dame-De-Grace (CA); Gary Edwards, Montreal (CA)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: LALLEMAND USA, INC., Montreal (CA)

(21) Appl. No.: 11/977,568

(22) Filed: Oct. 25, 2007

Related U.S. Application Data
(60) Provisional application No. 60/854,795, filed on Oct. 27, 2006.

(51) Int. Cl. A23L 1/28; C12N 1/16; A23L 1/303

(52) U.S. Cl. 426/62; 435/255.4; 435/255.1; 426/73

(57) ABSTRACT

The present invention relates to novel yeast. More specifically, a novel yeast that is enriched for Vitamin D. In one aspect, the invention comprises a yeast that retains its gassing power after UV irradiation and that can be used to produce breads and other baked products with significant levels of vitamin D, in particular Vitamin D2. The invention also relates to a method of producing a novel D2 enriched yeast as well methods of using the novel yeast of the invention.

The present application claims priority to U.S. provisional application entitled “Novel Vitamin D2 Yeast Preparation, A Method For Producing The Same, And Use Thereof” having Ser. No. 60/854,795 filed on Oct. 27, 2006, and herein incorporated by reference.

### FIELD OF THE INVENTION

The present invention relates to novel yeast. More specifically, a novel yeast that is enriched for Vitamin D. In one aspect, the invention comprises a yeast that retains its gassing power after UV irradiation and that can be used to produce breads and other baked products with significant levels of vitamin D, in particular Vitamin D2. The invention also relates to a method of producing a novel D2 enriched yeast as well methods of using the novel yeast of the invention.

### BACKGROUND OF THE INVENTION

Vitamin D is a hormone precursor essential to maintaining normal levels of calcium and phosphorous in the blood. The human body is capable of producing sufficient vitamin D, specifically Vitamin D3 (cholecalciferol), during exposure of the skin to the UV rays found in sunlight. As a consequence of lifestyle or through conscious choice, many individuals receive inadequate exposure to sunlight and consequently do not produce adequate quantities of Vitamin D. The availability of Vitamin D through dietary sources has therefore become increasingly important. Traditionally, the primary source of Vitamin D in the diet has been fortified milk. Lower per capita consumption of milk, however, has resulted in a lack of proper levels of Vitamin D in much of the population.

In addition to milk and other dairy products, breads have been seen as a cost-effective way to deliver fortified vitamins, including Vitamin D to a wide range of consumers. Generally, bakers have to go through a series of expensive and cumbersome steps to add several nutrients to their formulations. Generally, Vitamin D3 has been one such nutrient. Unfortunately due to its animal origin, D3 is not an acceptable additive for the entire population.

Although Vitamin D's role as a regulator of serum calcium and phosphorous is well established, more recent work is shedding light on many other health benefits associated with adequate levels of Vitamin D. These include:

Cell differentiation: Cells dividing rapidly are proliferating. Differentiation reduces proliferation and is critical to confer specific functions for different cells. Proliferation is necessary for growth and wound healing but if uncontrolled can lead to mutations and cancer. The active form of vitamin D inhibits proliferation and stimulates cell differentiation;

Immunity: Vitamin D is a potent immune system modulator and may inhibit autoimmunity;

Insulin Secretion: The VDR (vitamin D receptor) is expressed by insulin: secreting cells of the pancreas. Animal studies suggest that active Vitamin D plays a role in insulin secretion during conditions of high insulin demand. Limited data in humans suggests that vitamin D may have an effect on insulin secretion and glucose tolerance in type 2 diabetes; and

Blood Pressure: Adequate levels of Vitamin D may play a role in some forms of hypertension by reducing the risk of high blood pressure.

Vitamin D occurs in multiple forms including but not limited to D1, D2, D3, D4, and D5. Commercially, Vitamin D3 is the form found in fortified milk and has been commonly commercially derived from lanolin (sheep) or fish. In addition to Vitamin D3, Vitamin D2 has also shown to be bioavailable, well absorbed and to possess an active role in bone mineralization in animals (Bioavailability of Vitamin D2 from irradiated mushrooms: an in vivo study. Jasinghe, V. J. et al, British Journal of Nutrition, 93: 951-955 (2005)).

Yeast (Saccharomyces in particular) is known to have a high nutritional value, in particular as a good source of Vitamin B. Brewer's yeast, for example, has been sold commercially as a human nutritional supplement for years. Other yeast like Torula, Candida and Kluyveromyces have also been used as a source of growth factors and vitamins, either as nutritional supplement for human use or/and as for animal feed. This group of products is known as Nutritional Yeast and consists of yeast biomass or pure, dead yeast cells (Chapter 6: Yeast Technology, in Microbial Technology, Henry J. Peppler (ed.), Reinhold Publishing Corporation (1967)).

Yeast, however, does not contain Vitamin D, per se, but a unique sterol, ergosterol, which has the property of being transformed into Vitamin D2 when illuminated with UV light.

Not only is UV light able to convert ergosterol to Vitamin D2 in yeast, UV light is well known to inactivate and kill many microbes including viruses, bacteria, molds and yeast (Wolfe R. L. Ultraviolet disinfection of portable water, current technology and research needs. Environ Sci Technol 1990; 24(6):768-73; Hijnen W. A. M., Beerendonk E. F., and Medema G. J. Inactivation credit of UV radiation for viruses, bacteria and protozoan (oo)cysts in water: A review. Water Res. 2006; 40:3-22; Green C. F., Scarpino P. V., Jensen P., Jensen N. J., and Gibbs S. G. Disinfection of selected Aspergillus spp using ultraviolet germicidal irradiation. Can. J. Microbiol 2004; 50:221-224). Specifically, electromagnetic radiation with wavelengths ranging from 240 to 280 nm (ultraviolet) is well established as an effective agent for microorganism inactivation. Ultraviolet rays inactivate microorganisms by causing irreparable damage to their nucleic acid. The formation of pyrimidine dimers and other photoproducts of nucleic acids, inhibit DNA replication and transcription and hence prevent the cell or virus from multiplying. Consequently, irradiation of compositions containing live yeast generally results in inactivating (killing) the yeast, making it difficult or impossible for one to use irradiated yeast in certain commercial applications. Irradiated, inactivated (i.e. dead) yeast was sold for many years as animal feed before Vitamin D3 became less expensive and more popular as a feed supplement. In fact, UV irradiation is so effective in killing micro-organisms it has become widely used for drinking water disinfection and wastewater treatment in recent years (Kruithol J. C., Van der Leer R. C., Hijnen W. A. M. Practical experiences with UV disinfection in The Netherlands. J Water SRT-AQUA 1992; 41(2), 88-94; Liberti L., Notarnicola M., Lopez A. and Petruzzelli D. Advanced treatment for municipal wastewater reuse in agriculture. UV disinfection: bacteria inactivation, parasite removal and by-product formation. Desalination 2002; 152:315-324; Whitby G. E. and Palmateer G. The effect of UV transmission, suspended solids, wastewater mixtures and photoreactivation on microorganisms in wastewater treated with UV light. Water Sci Technol 1993; 27:379-386).

The present invention has resolved these problems. It was found that it is possible, contrary to that which was generally known, to produce yeast, specifically baker's yeast, enriched in vitamin D2, by irradiating the yeast. Rather than being inactive, the yeast of the invention kept most of its raising power, even after irradiation. This live, Vitamin D enriched, yeast can be used to fortify various baked goods (such as breads) and can help simplify operations at the bakery itself.

### DESCRIPTION OF THE FIGURES

FIG. 1 is a depiction of a UV photo-bioreactor useful in irradiating the yeast of the invention.

FIG. 2 is a depiction of another UV photo-bioreactor useful in irradiating the yeast of the invention.

FIG. 3 is a graph depicting Vitamin D2 enrichment kinetics in yeast during UV irradiation at different wavelengths

### SUMMARY OF THE INVENTION

This invention relates to yeast compositions enriched in Vitamin D. More specifically, the invention relates to a composition comprising yeast that has been enriched in Vitamin D2. In one aspect, the invention relates to compositions comprising live yeast that has been UV treated to transform the yeast's ergosterol content into Vitamin D2.

In one aspect of the invention the Vitamin D enhanced yeast maintains most of its raising power after UV treatment. In a further aspect of the invention the Vitamin D enhanced yeast maintains at least 50% of its raising power that was present prior to treatment by radiation.

In another aspect of the invention the Vitamin D content of the Vitamin D enhanced yeast is increase at least 10-fold and more preferably increased at least 50-fold.

In a further aspect of the invention the yeast is a baker's yeast strain of the genus Saccharomyces.

In another aspect, the invention contemplates compositions comprising Vitamin D enhanced yeast wherein the yeast is nutritional yeast. In one embodiment, the nutritional yeast is selected from the group consisting of Candida, Torula and Kluyveromyces.

In another aspect, the invention comprises a Vitamin D enriched yeast strain that is also enriched in minerals (Calcium, Zinc, Magnesium, Manganese and other minerals of physiological interest) and/or vitamins (Vitamins B family and other vitamins of physiological interest).

In another aspect, the invention comprises a composition wherein the Vitamin D enhanced yeast is in the form of cream yeast, compressed yeast, crumbled yeast, frozen yeast, freeze-dried yeast, active dry yeast or instant dry yeast. In one embodiment, the yeast of the invention is stabilized cream yeast.

In another aspect, the invention comprises a composition having Vitamin D enhanced yeast and further comprising enzymes useful in baking. In various embodiments the enzymes of interest are selected from the group consisting of amylases, xylanases, hemicellulases, cellulases, and lipases).

In a further aspect, the composition of the invention further comprises a Vitamin D enhanced yeast preparation that contains lactic acid bacteria. In one embodiment of the invention the Lactic Acid Bacteria are from the genus lactobacillus.

In another aspect, the invention comprises a Vitamin D enhanced yeast composition wherein the yeast has been inactivated by heat or other means.

A further aspect of the invention is the use of a Vitamin D enhanced yeast composition in the manufacture of breads, crackers, sports bars, biscuits, etc and other baked goods, as well as other functional foods and dietary food supplements.

The invention further contemplates the use of at least one of the preparations mentioned above as a nutrient or vitamin source for animal application.

The invention also contemplates the use of one or more of the aforementioned compositions as a nutrient or vitamin source in fermented beverages and fermented foods such as sauerkrauts.

The invention also contemplates a method for increasing the Vitamin D content of yeast comprising irradiating the yeast with UV radiation wherein the raising power of the yeast is substantially maintained.

### DETAILED DESCRIPTION OF THE INVENTION

Vitamin D is essential for good health for both humans and other animals. Humans are capable of producing Vitamin D, namely Vitamin D3 when exposed to the UV radiation in sunlight. In addition, Vitamin D is available through dietary means, most specifically fortified milk. With individuals spending less time in the sun and reduced milk consumption especially among adults these sources of Vitamin D have become insufficient to provide for the Vitamin D levels necessary for good health.

Fortified breads and cereals have been an auxiliary source of various vitamins and minerals in the diet, however the process requires bakers to go through a series of expensive and cumbersome steps to these nutrients to their formulations. Vitamin D2 has been one such nutrient. Commercially available Vitamin D is both expensive and isolated from animal sources making it an unacceptable additive for at least a portion of the population.

What is needed therefore is a way of producing Vitamin D that is efficient, inexpensive, wherein the Vitamin D does not come from animal sources and wherein the process is compatible with traditional baking.

The present invention solves these problems by providing for novel yeast: compositions wherein the yeast itself has an enhanced Vitamin D content. In one aspect of the invention, the yeast is enhanced for Vitamin D2. In one embodiment the yeast is enhanced for Vitamin D2 by irradiation with UV light.

In a further aspect of the invention the Vitamin D2 enhanced yeast maintains most of its raising power after UV treatment. More particularly, the invention contemplates a Vitamin D enhanced yeast composition wherein the yeast maintains at least 50% of its raising power present prior to treatment by radiation. In further embodiments the yeast maintains at least 60%, at least 70%, at least 75%, at least 80% and at least 85% of its raising power when compared to comparable non-irradiated yeast.

In another aspect of the invention, the Vitamin D content of the Vitamin D enhanced yeast is increase at least 10-fold and more preferably increased at least 50-fold. In further embodiments, the Vitamin D content of the yeast of the invention is increased at least 80-fold, at least 100-fold, at least 500-fold, at least 800-fold, at least 1.000-fold, at least 5.000-fold, at least 8.000-fold and at least 11.000-fold when compared to comparable non-irradiated yeast.

The Vitamin D enhanced yeast can be in any number of forms including cream yeast, compressed yeast, crumbled yeast, frozen yeast, freeze-dried yeast, active dry yeast or instant dry yeast. In one embodiment, the yeast of the invention is a stabilized cream yeast, in particular the stabilized cream yeast described in co-pending U.S. patent application Ser. No. 11/474,058 which is hereby incorporated by reference.

The Vitamin D enhanced yeast of the invention may also be subject to additional processing after irradiation. For example the invention contemplates a Vitamin D enhanced yeast composition wherein the yeast has been inactivated by heat or other means.

Additionally, the invention contemplates compositions of Vitamin D enhanced yeast that also contain lactic acid bacteria. In one embodiment of the invention the lactic acid bacteria are from the genus Lactobacillus.

The present invention also contemplates a Vitamin D enriched yeast composition wherein the yeast is a high nitrogen, protein, activity or budding yeast. Such high activity or budding include, but are not limited to living yeast cells such as from the genera Saccharomyces, Kluyveromyces, and Torulaspora. In particular the invention contemplates a Vitamin D enriched yeast of the species Saccharomyces cerevisiae. The invention also comprises combinations of one or more yeast species.

Processing aids can be added to the compositions of the invention in such: an amount that the properties of the final product are improved when said compositions are added to the fermenting mixture or dough. As described below, the processing aids can be divided into nutrients, chemical additives and enzymes.

Nutrient components can include inorganic nitrogen (such as urea and nitrogen salts), organic nitrogen (such as yeast, yeast autolysate, yeast extract, or fermentation solubles), phosphrous (such as salts of nitrogen and phosphorous), minerals (as salts), and vitamins. Mineral processing aids can include but are not limited to calcium, zinc, magnesium, manganese and other minerals of physiological interest. Vitamin processing aids can include any vitamin of physiological interest, including but not limited to, the B family of vitamins.

Suitable chemical additives are oxidizing agents such as ascorbic acid, bromate and azodicarbonamide and/or reducing agents such as L-cysteine and glutathione. A preferred oxidizing agent often used for baking is ascorbic acid, which is added to the composition in such amounts that result in an amount between 5 and 300 mg per kg flour. Other suitable chemical additives are emulsifiers acting as dough conditioners such as diacetyl tartaric esters of mono/diglycerides (DATEM), sodium stearoyl lactylate (SSL) or calcium stearoyl lactylate (CSL), or acting as crumb softeners such as glycerol monostearate (GMS) or bile salts, fatty materials such as triglycerides (fat) or lecithin and others. Preferred emulsifiers are DATEM, SSL, CSL or GMS. Preferred bile salts are cholates, deoxycholates and taurodeoxycholates.

Suitable enzymes are starch degrading enzymes, arabinoxylan and other hemicellulose degrading enzymes, cellulose degrading enzymes, oxidizing enzymes, fatty material splitting enzymes, protein degrading enzymes. Preferred starch degrading enzymes are endo-acting amylases such as alpha-amylase and exo-acting amylases such as beta-amylase and glucoamylase. Preferred arabinoxylan degrading enzymes are pentosanases, hemicellulases, xylanases and/or arabinofuranosidases, in particular xylanases from Aspergillus or Bacillus species. Preferred cellulose degrading enzymes are cellulases (i.e. endo-1,4-beta-glucanases) and cellobiohydrolasesi in particular from Aspergillus, Trichoderma or Humicola species. Preferred oxidizing enzymes are lipoxygenases, glucose oxidases, sulfhydryl oxidases, hexose oxidases, pyranose oxidases and laccases. Preferred fatty material splitting enzymes are lipases, in particular fungal lipases from Aspergillus or Humicola species, and phospholipases such as phospholipase A1 and/or A2. Preferred protein degrading enzymes are endo-acting proteinases such as those belonging to the classes thiolproteases, metalloproteases, serine proteases and aspartyl proteases, as well as exo-acting proteinases, also referred to as peptidases, belonging to the class of aminopeptidases and carboxypeptidases. Additionally, microbial and plant proteases for producing free amino nitrogen from the proteins in grain can also be added.

The enzymes may originate from animal, plant or microbial origin and they may be obtained from these sources by classical processes known in the art, or, alternatively, they may be produced via recombinant DNA technology. A preferred production process comprises fermentation processes in which fungi, yeast or bacteria are grown and produce the desired enzymes, either inherently or as a result of genetic modification (recombinant DNA technology). These processes are well known in the art. Preferably, the enzymes are secreted by the micro-organisms into the fermentation broth. At the end of the fermentation process, the cell biomass is usually separated and, depending on the enzyme concentration in the broth, the latter may be concentrated further and optionally washed by known techniques such as ultrafiltration. Optionally, the enzyme concentrates or a mixture of such concentrates may be dried by known techniques such as spray drying.

The compositions of the invention may be applied to any number of uses. In one aspect the compositions of the invention may be used in baking and in particular commercial baking. The compositions of the invention may be used to manufacture any type of baked good including but not limited to breads, crackers, sports bars, biscuits and other baked goods.

Such Vitamin D enriched yeast preparations are not only of interest for the baking industry but are also applicable to portable alcohols (distilling), brewing, baking, fermented beverages in general and any fermentation process.

A further object of the invention is a novel process for the production of ethanol, comprising the direct addition or pitching of a Vitamin D enhanced yeast of the invention to a production fermentor, thereby obviating the need for a propagation step.

Additional uses include the manufacture of preparations as a nutrient or vitamin source. In one embodiment the Vitamin D enhanced yeast of the invention is used for animal application. In another embodiment the aforementioned compositions of the invention are used as a nutrient or vitamin source in fermented beverages and fermented foods such as sauerkrauts.

The invention also contemplates a method for increasing the Vitamin D content of yeast comprising irradiating the yeast with UV radiation wherein the raising power of the yeast is substantially maintained. The UV radiation used can be of any wavelength but preferably is between 253 and 366 nanometers. Other spects of the invention include using UV wavelengths of between (and including) 255 nm to 270 nm, 270 nm to 290 nm, 290 nm to 310 nm, 310 nm to 330 nm, 330 to 350 and 350 to 366 nm. In one embodiment, the UV radiation used has a wavelength of about 254 nm. In another embodiment, the UV radiation used has a wavelength of 302 nm. In yet another embodiment the UV radiation used has a wavelength of about 365 nm.

The invention is not restricted to any specific type of yeast and, in particular, the invention is not restricted to a Vitamin D enhanced yeast composition wherein the yeast is Saccharomyces. In fact, it would be obvious to one ordinary skill in the art that the invention would include all types of used in commercial baking and fermentation processes.

### EXAMPLES

### Example 1

### Commercial Production of Yeast

The production of yeast for use in commercial fermentation is, in itself, a multi-step process. Generally, manufacturers of yeast for the baking industry have to produce yeast that must be packaged, stored and shipped in large quantities in a manner that guarantees the purity and viability of the final yeast product.

Baker's yeast production often starts with a pure culture tube or frozen vial of the appropriate yeast strain. This yeast serves as the inoculum for the pre-pure culture tank, a small pressure vessel where seed is grown in medium under strict sterile conditions. Following growth, the contents of this vessel are transferred to a larger pure culture fermentor where propagation is carried out with some aeration, again under sterile conditions. These early stages are conducted as set-batch fermentations. In set-batch fermentation, all the growth media and nutrients are introduced to the tank prior to inoculation.

From the pure culture vessel, the grown cells are transferred to a series of progressively larger seed and semi-seed fermentors. These later stages are conducted as fed-batch fermentations. During fed-batch fermentation, molasses, phosphoric acid, ammonia and minerals are fed to the yeast at a controlled rate. This rate is designed to feed just enough sugar and nutrients to the yeast to maximize multiplication and minimize the production of alcohol. In addition, these fed-batch fermentations are not completely sterile. It is not economical to use pressurized tanks to guarantee sterility of the large volumes of air required in these fermentors or to achieve sterile conditions during all the transfers through the many pipes, pumps and centrifuges. Extensive cleaning of the equipment, steaming of pipes and tanks and filtering of the air is practiced to insure as aseptic conditions as possible.

At the end of the semi-seed fermentation, the contents of the vessel are pumped to a series of separators that separate the yeast from the spent molasses. The yeast is then washed with cold water and pumped to a semi-seed yeast storage tank where the yeast cream is held at approximately 34 degrees Fahrenheit until it is used to inoculate the commercial fermentation tanks. These commercial fermentors are the final step in the fermentation process and are often referred to as the final or trade fermentation.

Trade fermentations are carried out in large fermentors with working volumes up to 50,000 gallons. To start the commercial fermentation, a volume of water, referred to as set water, is pumped into the fermentor. Next, in a process referred to as pitching, semi-seed yeast from the storage tank is transferred into the fermentor. Following addition of the seed yeast, aeration, cooling and nutrient additions are started to begin the 15-20 hour fermentation. At the start of the fermentation, the liquid seed yeast and additional water may occupy only about one-third to one-half of the fermentor volume. Constant additions of nutrients during the course of fermentation bring the fermentor to its final volume. The rate of nutrient addition increases throughout the fermentation because more nutrients have to be supplied to support growth of the increasing cell population. The number of yeast cells increase about five- to eight-fold during this fermentation.

Air is provided to the fermentor through a series of perforated tubes located at the bottom of the vessel. The rate of airflow is about one volume of air per fermentor volume per minute. A large amount of heat is generated during yeast growth and cooling is accomplished by internal cooling coils or by pumping the fermentation liquid, also known as broth, through an external heat exchanger. The addition of nutrients and regulation of pH, temperature and airflow are carefully monitored and controlled by computer systems during the entire production process. Throughout the fermentation, the temperature is kept at approximately 86 degrees Fahrenheit and the pH is generally in the range of 4.5-5.5.

At the end of fermentation, the fermentor broth is separated by nozzle-type centrifuges, washed with water and re-centrifuged to yield a yeast cream with a solids concentration of 15 to 24%, and often in the 18% range. The yeast cream is cooled to about 45 degrees Fahrenheit and stored in a separate, refrigerated stainless steel cream tank. Cream yeast can be loaded directly into tanker trucks and delivered to customers equipped with an appropriate cream yeast handling system. Alternatively, the yeast cream can be pumped to a plate and frame filter press or a rotary vacuum filtration system and dewatered to a cake-like consistency containing 27-33% yeast solids. This press cake yeast is crumbled into pieces and packed into 50-pound bags that are stacked on a pallet. The yeast heats up during the pressing and packaging operations and the bags of crumbled yeast must be cooled in a refrigerator for a period of time with adequate ventilation and placement of pallets to permit free access to the cooling air. Palletized bags of crumbled yeast are then distributed to customers in refrigerated trucks. Cream yeast can also be further processed into dried yeast (92-97% solids) by using a fluid bed dryer or similar types of dryers.

### Example 2

### UV Irradiation of Active Baker's Yeast Cream

A. Activity

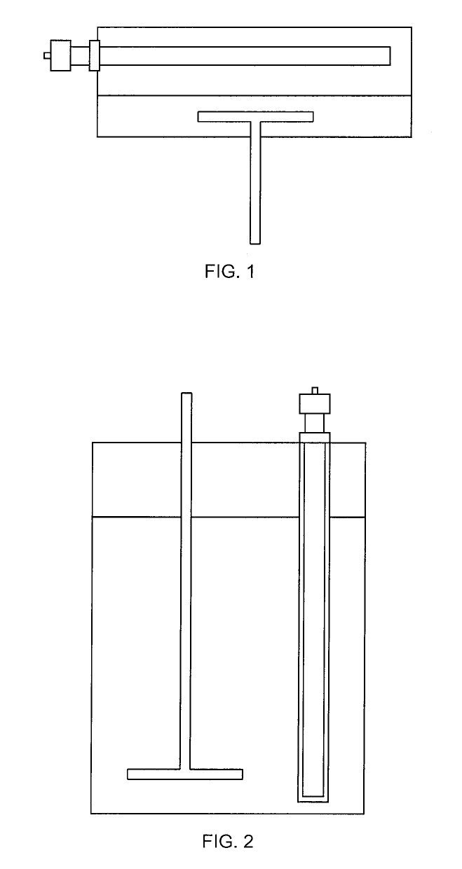

Commercial yeast cream with about 20% solids was directly irradiated using a lab scale UV photo-bioreactor as illustrated in FIG. 1. The photo-bioreactor set-up included a UV lamp, a shallow rectangular plastic container and a magnetic stirrer. The center of the photo-bioreactor set-up was the 8 W UV lamp from UVP with three switchable UV tubes-shortwave (254 nm), midrange (302 nm) and longwave (365 nm). Initially the midrange wavelength was used (302 nm). The UV lamp was installed 5-10 cm above the cream yeast surface and was never in contact with the yeast cream. Since the yeast cream is nearly opaque to UV light, it is necessary to stir the yeast cream during the irradiation so that all yeast cells would be moved to the surface and all the molecules of provitamins (ergosterol) in the yeast cells would be submitted to the UV irradiation. The shallow container was used to achieve a thin layer of yeast cream so that yeast cells could be transported to the surface and got irradiated more frequently, with an intention to achieve higher vitamin D2 conversion efficiency in yeast. Thirty (30) mL commercial yeast cream was loaded in the rectangular container and continuously irradiated for 1 hour. During the irradiation, the yeast cream was mixed continuously. The experiments were conducted at room temperature. After 1 hour irradiation, the vitamin D2 content in yeast was increased from 2,370 to 1,980,000 IU/100 g (dry weight), an increase of 835 times; the sweet dough activity of the semi-seed yeast was decreased by about 10% only, from 456 cc to 424 cc of CO2. Therefore, the vitamin D2 in yeast was enriched dramatically while most of the yeast baking activity was retained after 1 hour UV irradiation.

The vitamin D2 analyses were done in Covance Laboratories Inc. HPLC was used for the vitamin D2 determination with an official method (Official Methods of Analysis of AOAC INTERNATIONAL (2000) 17^{th} Ed., AOAC INTERNATIONAL, Gaithersburg, Md., USA, Official Methods 982.29).

The sweet dough activity of the yeast was measured in a SJA Fermentograph. The ingredients for the sweet dough are shown in Table 1. The dough was incubated in the SJA Fermentograph at 35° C. for 60 minutes and the total gassing volume achieved was expressed as yeast sweet activity.

TABLE 1
Recipe for the sweet dough

| | | |
|---|---|---|
| Flour (Three Stars) | 300 | g |
| Granulated Sugar | 50 | g |
| Salt | 5 | g |
| Shortening | 50 | g |
| Yeast Cream | 24.5 | g |
| Tap Water at 35° C. | 137.5 | g |

B. Effect of UV Wavelength

To examine the effect of the wavelength on yeast vitamin D2 enrichment, the UV irradiation of the yeast cream was carried out substantially as in Example 1 above using three different wavelengths: shortwave (254 nm), midwave (302 nm) and longwave (365 nm), respectively. At each wavelength, two different irradiation time (2 and 4 hours) was used in order to evaluate the effect of irradiation time on vitamin D2 enrichment. The experiments were conducted at room temperature.

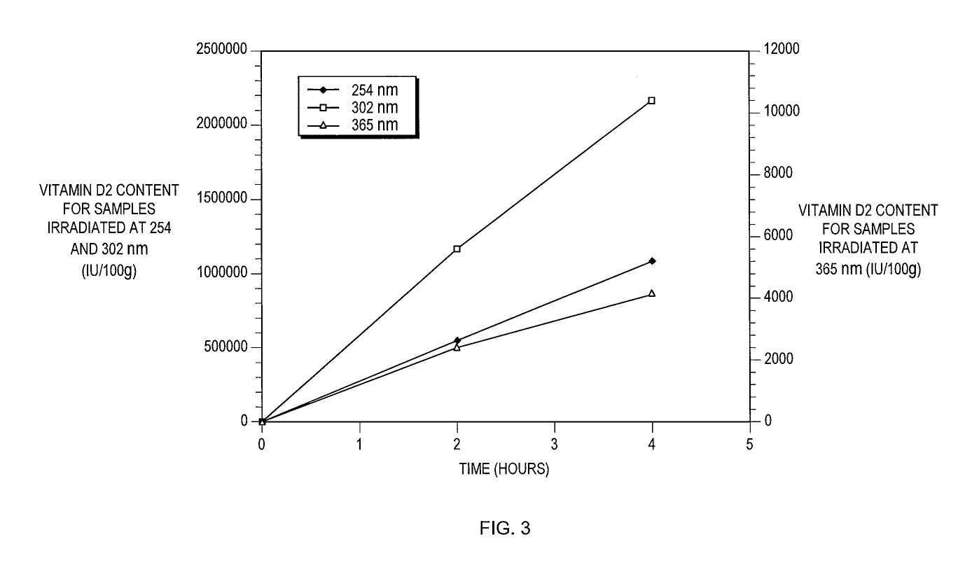

In the experiments with the surface UV irradiation photo-bioreactor, 7 dry yeast samples (oven-dried) were produced and sent to Covance for vitamin D2 analysis. The vitamin D2 contents for the 7 yeast samples are shown in Table 2. The control sample was prepared from the yeast cream before UV irradiation. The other 6 yeast samples were obtained by irradiating the yeast cream for 2 or 4 hours at three different wavelengths (254, 302 and 365 nm). For all 6 samples, the size of the yeast cream for irradiation was 400 mL. Table 2 shows that the UV wavelength strongly influenced the vitamin D2 enrichment in yeast. Once again, it was found that vitamin D2 could be dramatically enriched in yeast at UV wavelengths 254 and 302 nm. Compared to the yeast samples irradiated at 254 nm, much higher vitamin D2 content was achieved with the yeast samples irradiated at 302 nm wavelength. Interestingly, it was observed that the yeast samples irradiated at 365 nm gave much lower vitamin D2 results, suggesting that the wavelength 365 nm less effective in enriching vitamin D2 in yeast. Therefore, the ideal wavelength for vitamin D2 yeast production is 302 nm. FIG. 3 is a graphical depiction of the data below reflecting the zero-order kinetics of the reaction.

TABLE 2
Effects of UV wavelength and irradiation time on vitamin
D2 enrichment (IU = vitamin D2 international unit).

| UV | Irradiation Time | |
|---|---|---|
| Wavelength | 2 hours | 4 hours |
| 254 nm | 545,000 IU/100 g | 1,090,000 IU/100 g |
| 302 nm | 1,160,000 IU/100 g | 2,160,000 IU/100 g |
| 365 nm | 2,350 IU/100 g | 4,240 IU/100 g |

| | |
|---|---|
| Control Yeast | 75 IU/100 g |
| (before irradiation) | |

### Example 3

### UV Irradiation of Large Scale Batches

In another experiment, the irradiation of yeast cream was carried out in the UV photo-bioreactor as illustrated in FIG. 2, which was designed to be capable of processing larger volume. Fifteen (15) liters of the commercial yeast cream was loaded in the 20 liters photo-bioreactor, which was equipped with a 14-Watts UV lamp with UV rays of 254 nm wavelength from Atlantic Ultraviolet Corporation. The UV lamp was immersed in the yeast cream through a quartz sleeve. Vigorous agitation was provided with a lightning agitator to move yeast cells to the irradiation zone frequently and to maintain high transmission of UV rays by preventing potential fouling around the quartz sleeve. The 15 liters of yeast cream was continuously mixed and irradiated for 8 hours at room temperature. After 8 hours of irradiation, the vitamin D2 content in yeast was increased from 2,370 to 198,000 IU/100 g (dry weight), an increase of 84 times; the sweet dough activity of the yeast was decreased from 600 to 550 cc, a decrease of a little more than 10%. Again, the vitamin D2 in yeast was enriched dramatically while most of the yeast baking activity was retained. As expected, the yeast vitamin D2 content achieved in this experiment was much less than that achieved in the previous experiment, due to much larger processing volume of the yeast cream.

### Example 4

### UV Irradiation of Inactive Yeast

UV irradiation experiments were also conducted with commercial inactive yeast produced in Lallemand Denmark (product code-213625, lot#5196D). The inactive dry yeast was dissolved in tap water to make a cream with 10% solids. 30 mL of the obtained yeast cream was directly irradiated for 1 hour at room temperature using the photo-bioreactor set-up shown in FIG. 1. As a result of the UV irradiation, the vitamin D2 content in yeast was increased from 324 to 3,810,000 IU/100 g (dry weight), an dramatic enrichment of 11759 times compared to the control. Therefore, the UV irradiation process was also suitable for inactive yeast.

### Example 5

### Standardization of Commercial Vitamin D2 Enriched Yeast Production

In order to achieve consistent vitamin D2 content in yeast in the production of the vitamin D enriched active baker's yeast, it was recommended to standardize the production by blending certain portion of high strength vitamin D2 enriched yeast with regular commercial yeast cream. The high strength vitamin D2 enriched yeast can be prepared in the forms of cream, cake and IDY (Instant Dry Yeast).

Table 3 shows the yeast usages for the bread samples made with a regular yeast cream and a vitamin D2 enriched active yeast cream. The yeast vitamin D2 content in the vitamin D2 enriched active yeast cream was about 1,600,000 IU/10 g (high strength). For both white and whole wheat breads, the usage of the vitamin D2 enriched active yeast cream was about 8% of total yeast used. With this percentage, a vitamin D2 content of 400 IU per 50 gram bread was achieved. By consuming 50 g or 2 slices of such breads per day, people are able to meet their RDA for vitamin D (400 IU). Bakers usually are content providing partial vitamin D RDA since they want to be sure it is not too much. If the bakers would like to provide 20% of the RDA per 2 slices of bread (daily bread serving), then the portion of the vitamin D2 enriched active cream yeast would be about 1.6% of total yeast used. On the other hand, if the bakers would like to provide only 10% of the RDA per 2 slices of bread, then the portion of the vitamin D2 enriched active cream yeast would be about 0.8% of total yeast used. So by blending about 0.8% of the vitamin D enriched active cream yeast (UV irradiated to achieve 1,600,000 IU/100 g vitamin D2 in yeast) with 99.2% of the regular cream yeast, the vitamin D enriched active cream yeast targeting 10% of RDA per daily bread serving could be readily produced. For example, by blending 800 liters of the UV irradiated cream yeast with 99,200 liters of the regular cream yeast, we can make 100,000 liters of vitamin D enriched active yeast cream targeting 10% of RDA for vitamin D. In this case, it can be envisioned that we can design and construct a small photo-bioreactor of about 1000 liters to produce the 800 liters of UV irradiated cream yeast. The small photo-bioreactor means small capital investment as well as operating cost. The standardized vitamin D2 enriched active yeast cream would also expect to be as active as the regular liquid yeast. So the vitamin D2 enriched active yeast cream would be marketed as a value-added product.

Another option is to produce a large batch of the high strength vitamin D2 enriched yeast in the form of instant dry yeast (IDY). Then the vitamin D2 enriched active yeast cream with certain vitamin D2 content could be readily produced by blending certain amount of the vitamin D2 enriched IDY with the regular yeast cream. Due to the much longer shelf life of the IDY, the advantage for this option is that we can produce a larger batch of the high strength vitamin D2 enriched yeast, which can be used for longer time. The disadvantage for this option is that we need a much larger UV photo-bioreactor to irradiate the yeast cream before drying so that we can process the batch in a reasonable time.

TABLE 3
Yeast usages for the 4 breads made with regular cream
yeast and vitamin D enriched active cream yeasts.

| | Yeast Samples | | |
|---|---|---|---|
| | | | Percentage of |
| | Regular | Vitamin D | Vitamin D |
| | Cream | Enriched Active | Enriched Active |
| | Yeast | Cream Yeast | Cream Yeast |
| Bread Samples | (g) | (g) | (%) |
| #1. White Bread with | 48 | 0 | 0 |
| Regular Cream Yeast | | | |
| #2. White Bread with | 44.25 | 3.75 | 7.8 |
| Vitamin D2 Yeast 1 | | | |
| #3. Whole Wheat Bread | 48 | 0 | 0 |
| with Regular Cream | | | |
| Yeast | | | |
| #4. Whole Wheat Bread | 44.0 | 4.0 | 8.3 |
| with Vitamin D2 | | | |
| Yeast 1 | | | |

### Example 6

### Bread Manufacturing

To demonstrate the effectiveness of treated bakers' yeast in producing bread with high levels of vitamin D, two bread formulations (white and whole wheat) were tested with four yeast samples (one control plus three vitamin D enriched yeasts). As a result, eight bread samples were prepared in total. Detailed dough formulations for the eight bread samples are shown in Table 4. Two slices of bread or 50 g is usually regarded as daily bread serving. For the breads made with vitamin D enriched yeasts, the amount of vitamin D2 enriched yeast required in the dough to achieve a vitamin D content of 400 IU per 50 gram bread was calculated based on the initial vitamin D content in the vitamin D enriched yeast samples. In the calculations, it was assumed that 350 g of bread could be made from 400 g dough.

After standard mixing, the dough temperature should be between 24 and 28° C. The dough was given an intermediate proof of 15 minutes then scaled at 400 g dough pieces, which were rounded then sheeted and molded in a Nussex moulder. The shaped dough pieces were placed in baking pans and proofed at 112° F. and 89% R.H. until reaching a 97 mm height. Then they were baked in a National oven for 17 minutes at 440° F. (226.7° C.). The breads were frozen before sending for vitamin D2 analysis.

To evaluate the vitamin D2 content in the bread, the 8 bread samples as described above were sent to an outside lab for vitamin D2 analysis. The first tests were done after the bread samples have been stored at room temperature for about 4 days. To examine if any loss of vitamin D2 occurred during bread storage at room temperature, the second vitamin D2 analyses were done with 14 days old bread samples. HPLC (Silliker AOAC method) has been used for vitamin D2 determination.

TABLE 4

| | Bread Type | | | | | | | |
|---|---|---|---|---|---|---|---|---|
| | White Bread | | | | Whole Wheat Bread | | | |
| Yeast | | Vit D | Vit D | Vit D | | Vit D | Vit D | Vit D |
| Samples | Control | Yeast 1 | Yeast 2 | Yeast 3 | Control | Yeast 1 | Yeast 2 | Yeast 3 |
| Code # | 1 | 2 | 3 | 4 | 5 | 6 | 7 | 8 |
| Mixer # | 1 | 2 | 1 | 2 | 1 | 2 | 1 | 2 |
| Labels | RED | ORANGE | BLUE | GREEN | PURPLE | YELLOW | 2× | 2× |
| | | | | | | | RED | ORANGE |
| White | 1000 | 1000 | 1000 | 1000 | 0 | 0 | 0 | 0 |
| Flour (g) | | | | | | | | |
| Whole Wheat | 0 | 0 | 0 | 0 | 1000 | 1000 | 1000 | 1000 |
| Four (g) | | | | | | | | |
| Water (g) | 610 | 610 | 630 | 630 | 730 | 730 | 750 | 750 |
| Dextrose (g) | 70 | 70 | 70 | 70 | 70 | 70 | 70 | 70 |
| Vegetable Oil | 30 | 30 | 30 | 30 | 30 | 30 | 30 | 30 |
| (g) | | | | | | | | |
| Salt (g) | 20 | 20 | 20 | 20 | 20 | 20 | 20 | 20 |
| CAP (g) | 2.5 | 2.5 | 2.5 | 2.5 | 2.5 | 2.5 | 2.5 | 2.5 |
| Fermaid C | 0.625 | 0.625 | 0.625 | 0.625 | 0.625 | 0.625 | 0.625 | 0.625 |
| (g) | | | | | | | | |
| SSL (g) | 3.75 | 3.75 | 3.75 | 3.75 | 3.75 | 3.75 | 3.75 | 3.75 |
| Control | 48 | 44.25 | 0 | 0 | 48 | 44 | 0 | 0 |
| Yeast Cream(g) | | | | | | | | |
| Vitamin D | 0 | 6.30 | 0 | 0 | 0 | 4 | 0 | 0 |
| Yeast 1 (g) | | | | | | | | |
| Vitamin D | 0 | 0 | 0.82 | 0 | 0 | 0 | 0.88 | 0 |
| Yeast 2 (g) | | | | | | | | |
| Vitamin D | 0 | 0 | 0 | 2.19 | 0 | 0 | 0 | 2.34 |
| Yeast 3 (g) | | | | | | | | |
| Fresh | 0 | 0 | 30 | 30 | 0 | 0 | 30 | 30 |
| Compressed | | | | | | | | |
| Yeast (g) | | | | | | | | |
| Total Dough | 1784.88 | 1787.43 | 1787.70 | 1789.07 | 1904.88 | 1904.88 | 1907.76 | 1909.22 |
| Weight (g) | | | | | | | | |
| Total Bread | 1561.77 | 1564.00 | 1564.23 | 1565.43 | 1666.77 | 1666.77 | 1669.29 | 1670.56 |
| Weight (g) | | | | | | | | |
| Target Vit D | 6.25 | 672 | 400 | 400 | 5.86 | 400 | 400 | 400 |
| per 50 g Bread | | | | | | | | |
| (IU) | | | | | | | | |

Table 5 shows the vitamin D2 contents of the 8 prepared bread samples after 4 and 14 days storage at room temperature. It was successfully demonstrated that using the vitamin D2 enriched yeasts could dramatically enrich the vitamin D2 in the bread. Compared to the control bread, the breads made with vitamin D2 enriched yeasts gave much higher vitamin D2 content. These results also showed that no significant vitamin D2 losses occurred during the bread making process and as a result, high vitamin D2 recovery efficiency has been achieved. The high vitamin D recovery efficiency suggests the vitamin D2 in the yeast was not susceptible to the high temperature (227° C.) baking process.

During storage at room temperature, breads come to contact with oxygen and light. There was a concern that the vitamin D2 in the breads might not be stable due to the potential oxidation and photochemical reactions. However, the bread vitamin D2 shelf life studies did not support this speculation. As shown in Table. 5, the vitamin D2 contents of the 14 days old breads were similar to those of 4 days old breads. There were no significant vitamin D losses after the breads have been stored at room temperature for 14 days (2 weeks). Therefore, the vitamin D2 in the bread was stable to storage. This observation is important since nowadays the bread shelf life could be up to 14 days. The 14 days results assure bakers that the vitamin D2 is stable and available for at least 2 weeks in the breads. In addition, the 14 days vitamin D2 results also confirmed the 4 days results.

TABLE 5
Vitamin D2 contents of the 8 bread samples after 4 and 14 days storage at room temperature

| | Theoretical | Vitamin D2 | Vitamin D2 |
|---|---|---|---|
| | Vitamin D2 | Content of 4 Days | Content of 14 Days |
| | Content in the | Old Bread at | Old Bread at |
| Bread Samples | Bread | Room Temperature | Room Temperature |
| #1. White Bread with | 12 IU/100 g Bread | <20 IU/100 g bread | <20 IU/100 g bread |
| Control Cream Yeast | | | |
| #2. White Bread with | 1344 IU/100 g Bread | 1410 IU/100 g bread | 1360 IU/100 g bread |
| Vitamin D2 Yeast 1 | | | |
| #3. White Bread with | 800 IU/100 g Bread | 845 IU/100 g bread | 869 IU/100 g bread |
| Vitamin D2 Yeast 2 | | | |
| #4. White Bread with | 800 IU/100 g Bread | 947 IU/100 g bread | 777 IU/100 g bread |
| Vitamin D2 Yeast 3 | | | |
| #5. Whole Wheat Bread with | 12 IU/100 g Bread | <20 IU/100 g bread | <20 IU/100 g bread |
| Control Cream Yeast | | | |
| #6. Whole Wheat Bread with | 800 IU/100 g Bread | 799 IU/100 g bread | 823 IU/100 g bread |
| Vitamin D2 Yeast 1 | | | |
| #7. Whole Wheat Bread with | 800 IU/100 g Bread | 783 IU/100 g bread | 756 IU/100 g bread |
| Vitamin D2 Yeast 2 | | | |
| #8. Whole Wheat Bread with | 800 IU/100 g Bread | 900 IU/100 g bread | 858 IU/100 g bread |
| Vitamin D2 Yeast 3 | | | |

### Example 7

### Manufacturing of Pizza Dough

In example 6, lab breading making trials using vitamin D enriched baker's yeast were carried out. It was successfully demonstrated that the vitamin D in the breads could be dramatically enriched by using the vitamin D enriched yeasts. Compared to the control bread made with regular baker's yeast, the breads made with vitamin D enriched yeasts gave much higher vitamin D content. The vitamin D in the yeast retained well in the breads and a very good vitamin D recovery efficiency was achieved, suggesting vitamin D in the yeast was not susceptible to the high temperature (227° C.) during baking process. The experimental results also showed good keepability of the vitamin D in the breads. No significant vitamin D losses were observed after the breads have been stored at room temperature for 2 weeks.

To further validate the vitamin D baker's yeast concept, industrial trials (dough for pizza crust) have been carried out at a commercial bakery using vitamin D enriched baker's yeast. The vitamin D enriched baker's yeast used in the industrial trials was in liquid form containing 15 IU/g vitamin D2. 24 lb of such vitamin D2 enriched liquid yeast was used per 226 lb flour or per 385 lb dough to deliver about 33% of the vitamin RDA per serving (The serving size was 144 g of the pizza crust). 6 inch pizza crust was produced and was bulk proofed for 24 hours. All the pizza crusts were hot pressed and then partly baked. Then they were topped with the toppings and CO2 frozen at −68 F for 22 minutes. Both the test pizza crust samples (produced with the vitamin D enriched baker's yeast) and the control pizza crust samples (produced with regular baker's yeast) were sent to Covance Laboratories for vitamin D2 analysis. Based on the analysis results from Covance Laboratories (Table 6), the actual vitamin D2 content detected in the test pizza crust samples was very close to the theoretical (expected) vitamin D2 content. The vitamin D2 enriched yeast cream was prepared to target 33% of the vitamin D RDA per serving or 128 IU per serving. The actual vitamin D2 content per serving pizza crust detected was 127 IU or 32% vitamin RDA. In contrast, vitamin D2 content detected in the control pizza crust samples was less than 20 IU per serving, which was much lower than the test pizza crust samples. Like the previous lab bread tests, significant vitamin D enrichment in the bread was observed as a result of using vitamin D enriched yeast. Very good vitamin D recovery efficiency was also achieved for the commercial pizza dough tests.

TABLE 6
Comparison of vitamin D2 contents between
test and control pizza crust samples

| | Test Pizza | Control Pizza |
|---|---|---|
| | Crust Sample | Crust Sample |
| | with Vitamin D | with Regular |
| Samples | Enriched Yeast) | Yeast |
| Actual Vitamin D2 in Pizza | 127 | <20 |
| Crust (IU Per Serving = 144 g) | | |
| Yeast Cream Used in the | 24 lb Vitamin D | 24 lb Regular |
| Dough (lb) | Enriched Liquid | Liquid Baker's |
| | Baker's Yeast | Yeast |
| Total Weight of Dough (lb) | 385 | 385 |
| Vitamin D2 Content in Yeast | 15 | Trace |
| Cream(IU/g) | | |
| Theoretical (Expected) | 128 | Trace |
| Vitamin D2 Content in Pizza | | |
| Crust Per Serving = 144 g | | |

### Example 8

### Manufacturing of Pizza Dough

Following the successful commercial trial with pizza crust, second industry trial with the vitamin D enriched baker's yeast was conducted at another commercial bakery. The tests were for the production of hamburger buns. The vitamin D enriched baker's yeast used in the tests was in liquid form with a vitamin D2 content of 22 IU/g. 54 lb of such vitamin D2 enriched liquid yeast was used per 1000 lb flour or per 1769 lb dough to deliver about 10% of the vitamin RDA per serving (The serving size was 60 g of hamburger bun). Both the test hamburger bun samples (produced with the vitamin D enriched baker's yeast) and the control hamburger bun samples (produced with regular baker's yeast) were sent to Covance Laboratories for vitamin D2 analysis. Table 7 summarizes the analysis results for the hamburger bun samples. Based on the usage of the vitamin D enriched baker's yeast in the dough, the theoretical (expected) vitamin D2 content in test samples would be 40 IU per serving. As shown in the Table 7, the actual vitamin D2 content detected in two test hamburger bun samples were 43.4 and 44.6 IU per serving, respectively, which was very close to the theoretical value. Like the first industrial trial, once again very good vitamin D2 mass balance was demonstrated. The 2 test hamburger bun samples also gave very similar vitamin D2 content, so did the 2 control bun samples, showing good reproducibility for the replicates.

TABLE 7
Comparison of vitamin D2 contents between
test and control hamburger bun samples

Vitamin D2 Content

| Hamburger Bun Samples | IU/100 g | IU per Serving |
|---|---|---|
| Control hamburger bun | <20 | |
| sample 1 | | |
| Control hamburger bun | <20 | |
| sample 2 | | |
| Test hamburger bun | 72.4 | 43.4 |
| sample 1 | | |
| Test hamburger bun | 74.4 | 44.6 |
| sample 2 | | |

1. A composition comprising yeast that has been UV treated to transform its ergosterol content into Vitamin D2.

2. The composition of claim 1 wherein the yeast has retained most of its raising power after UV treatment.

3. The composition of claim 1 wherein the yeast is a baker's yeast strain of the genus Saccharomyces.

4. The composition of claim 1 wherein the yeast is a nutritional yeast.

5. The composition of claim 4 wherein the nutritional yeast is selected from the group consisting of the genus Candida, the genus Torula and the genus Kluyveromyces.

6. The composition of claim 1 wherein the yeast is a yeast strain enriched in minerals.

7. The composition of claim 6 wherein the minerals are selected from the group consisting of Calcium, Zinc, Magnesium, and Manganese.

8. The composition of claim 1 wherein the yeast is a yeast strain enriched in vitamins other than Vitamin D2.

9. The composition of claim 2 wherein the yeast is selected from the group consisting of cream yeast, stabilized cream yeast, compressed yeast, crumbled yeast, frozen yeast, freeze-dried yeast, active dry yeast and instant dry yeast.

10. The composition of claim 1 wherein the composition contains enzymes.

11. The composition of claim 10 wherein the enzymes are selected from the group consisting of amylases, xylanases, hemicellulases, cellulases and lipases.

12. The composition of claim 1 wherein the said yeast preparation contains lactic acid bacteria contributing to the flavor of bread.

13. The composition of claim 1 wherein the yeast has been inactivated by heat or other means.

14. A baked good produced using any one of the compositions of claims 1-13.

15. The baked good of claim 14 selected from the group consisting of breads, crackers, sports bars and biscuits.

16. A composition of any one of claims 1-13 wherein the composition is delivered to a non-human animal.

17. A fermented product produced using the compostions of any one of claims 1-13.

18. The fermented product of claim 17 wherein the fermented product is selected from the group consisting of a fermented beverage and a fermented food.

19. The fermented product of claim 18 wherein the fermented product is sauerkrauts.

20. A composition comprising yeast having an enhanced Vitamin D content.

21. The composition of claim 20 wherein the yeast is a baker's yeast strain of the genus Saccharomyces.

22. The composition of claim 20 wherein the yeast is a nutritional yeast.

23. The composition of claim 22 wherein the nutritional yeast is selected from the group consisting of the genus Candida, the genus Torula and the genus Kluyveromyces.

24. The composition of claim 20 wherein the yeast is a yeast strain enriched in minerals.

25. The composition of claim 24 wherein the minerals are selected from the group consisting of calcium, zinc, magnesium and manganese.

26. The composition of claim 20 wherein the yeast is a yeast strain enriched in vitamins other than Vitamin D2.

27. A method of increasing the Vitamin D content of yeast comprising irradiating a yeast composition wherein the yeast maintains substantially all of its raising power after irradiation.

28. The method of claim 27 wherein the Vitamin D content of the yeast is increased by at least 1,000%.

29. The method of claim 28 wherein the Vitamin D content of the yeast is increased by at least 8,000%.

30. The method of claim 29 wherein the Vitamin D content of the yeast is increased by at least 80,000%.
